(19) United States
(12) Patent Application Publication
(10) Pub. No.: US 2021/0298609 A1
(43) Pub. Date: Sep. 30, 2021

(54) APPARATUS FOR SIMULTANEOUSLY MEASURING HETEROGENEOUS BIOSIGNALS AND MEASUREMENT METHOD THEREOF

(71) Applicant: ATSENS CO., LTD., Seongnam-si (KR)

(72) Inventors: Jong-Ook JEONG, Seongnam-si (KR); Chang-Ho LEE, Seongnam-si (KR); Bang Won LEE, Yongin-si (KR)

(21) Appl. No.: 17/345,522

(22) Filed: Jun. 11, 2021

Related U.S. Application Data
(63) Continuation-in-part of application No. PCT/KR2019/017430, filed on Dec. 11, 2019.

(30) Foreign Application Priority Data
Feb. 27, 2019 (KR) 10-2019-0023028

(51) Int. Cl. A61B 5/0205; A61B 5/00; A61B 5/08; A61B 5/318

(52) U.S. Cl. CPC A61B 5/0205; A61B 5/0006; A61B 5/0816; A61B 2562/0214; A61B 5/7405; A61B 5/6833; A61B 5/318

(57) ABSTRACT

Provided is an apparatus for measuring biosignals. The apparatus includes: a multiplexer configured to receive a first biosignal and a second biosignal, and to output either the first biosignal or the second biosignal; an analog-to-digital converter configured to convert an analog signal that is an output of the multiplexer into a digital signal; and a processor configured to perform signal processing on an output of the analog-to-digital converter.

### BACKGROUND OF THE INVENTION

### Field of the Invention

The present disclosure relates to an apparatus for simultaneously measuring heterogeneous biosignals, and a method of simultaneously measuring heterogeneous biosignals. In particular, the present disclosure relates to an apparatus for simultaneously measuring an electrocardiogram signal and a respiratory signal, and a method of simultaneously measuring an electrocardiogram signal and a respiratory signal.

### Description of the Related Art

In a number of fields including the study of human sleep, different types of biosignals need to be measured together. For example, in the case of the study of sleep, electrocardiogram signals as well as respiratory signals are required to be measured for detection of sleep apnea.

However, a biosignal measurement apparatus realized in the form of a patch needs to be reduced in size so as to minimize inconvenience to a subject for a long-time measurement (e.g. a few days).

As an example of reducing the biosignal measurement apparatus in size, there is a way of sharing some of multiple constituents when heterogeneous biosignals are simultaneously measured.

U.S. Pat. No. 9,192,316 (SYSTEMS AND METHODS USING FLEXIBLE CAPACITIVE ELECTRODES FOR MEASURING BIOSIGNALS) discloses a biosignal measurement apparatus and method that are capable of sharing a measurement electrode for an electrocardiogram signal and a respiratory signal.

In U.S. Pat. No. 9,192,316, used is a signal isolator that is capable of making a high impedance signal and a low impedance signal separated by using impedance characteristics of an electrocardiogram signal and a respiratory signal. However, in this case, an electronic element, such as a transformer, which is relatively large in physical size is required, so it is expected to be difficult to reduce the measurement apparatus in size and realize the same in the form of a patch.

### SUMMARY OF THE INVENTION

Accordingly, the present disclosure has been made keeping in mind the above problems occurring in the related art, and the present disclosure is directed to providing an apparatus for simultaneously measuring heterogeneous biosignals, and a measurement method thereof, the apparatus being realized in the form of a patch reduced in size by sharing some of constituents when heterogeneous biosignals are simultaneously measured.

According to the present disclosure, there is provided an apparatus for measuring biosignals, the apparatus including: a multiplexer configured to receive a first biosignal and a second biosignal, and to output either the first biosignal or the second biosignal; an analog-to-digital converter configured to convert an analog signal that is an output of the multiplexer into a digital signal; and a processor configured to perform signal processing on an output of the analog-to-digital converter. In addition, the multiplexer and the analog-to-digital converter may be provided as follows: the multiplexer and the analog-to-digital converter are separate constituents; or the multiplexer and the analog-to-digital converter are contained in one integrated circuit.

Furthermore, the multiplexer may select and output either the first biosignal or the second biosignal, and may output the first biosignal and the second biosignal alternately in succession.

In addition, the processor may take a signal input to the processor, and may perform separation of the first biosignal or the second biosignal for output. Preferably, the processor performs signal processing by using a first filter when the first biosignal is input, and performs signal processing by using a second filter when the second biosignal is input.

In addition, the processor may add, to the first biosignal or the second biosignal, time information on the time when the first biosignal or the second biosignal is measured and may output the resulting biosignal.

Preferably, according to the present disclosure, the apparatus for measuring biosignals further includes: a memory configured to store therein the first biosignal or the second biosignal processed by the processor; and a transceiver configured to transmit the first biosignal or the second biosignal processed by the processor to an external device. In addition, according to the present disclosure, the apparatus for measuring biosignals may transmit the first biosignal or the second biosignal to the external device through the transceiver when a communication state using the transceiver is stable; or may store the first biosignal or the second biosignal in the memory when communication state is unstable. In addition, according to the present disclosure, the apparatus for measuring biosignals may transmit data stored in the memory to an external device through the transceiver when the communication state using the transceiver is changed from an unstable state to a stable state.

Furthermore, when it is determined that the first biosignal or the second biosignal is abnormal, the processor may add an event mark of abnormality to an abnormal part of the first biosignal or the second biosignal. In addition, when it is determined that both the first biosignal and the second biosignal are abnormal simultaneously or within a predetermined period of time, the processor may add respective event marks of complex abnormality both to an abnormal part of the first biosignal and to an abnormal part of the second biosignal.

In addition, according to the present disclosure, the apparatus for measuring biosignals may further include an alarm configured to output a visual or auditory warning signal, wherein the alarm may output the warning signal in a preset manner according to the event mark added to the first biosignal or the second biosignal.

Furthermore, according to the present disclosure, the apparatus for measuring biosignals may further include: a first sensing stage configured to measure the first biosignal by using at least one electrode; and a second sensing stage configured to measure the second biosignal by using at least one electrode, wherein the first sensing stage and the second sensing stage may share at least one electrode. Specifically, the first biosignal may be an electrocardiogram signal, the second biosignal may be a respiratory signal, and the first sensing stage and the second sensing stage may share the one electrode. Furthermore, the second sensing stage may include: a capacitive element connected to the electrode shared with the first sensing stage; and a capacitive sensing circuit connected to the capacitive element and configured to sense a capacitance value. Specifically, the capacitive element may use either a capacitor or a capacitor equivalent device such as an ESD protection device and a buzzer.

In addition, according to the present disclosure, the apparatus for measuring biosignals may further include: a first sensing stage configured to measure the first biosignal by using at least one electrode; and a second sensing stage configured to measure the second biosignal, wherein the first biosignal is an electrocardiogram signal, the second biosignal is a respiratory signal, and the second sensing stage is connected to a buzzer and measures the second biosignal by using a change in a capacitance value between the buzzer and a skin of a subject.

According to the apparatus for simultaneously measuring heterogeneous biosignals and the measurement method thereof of the present disclosure, the apparatus can be realized in the form of a patch reduced in size by sharing some of constituents when heterogeneous biosignals are simultaneously measured.

### BRIEF DESCRIPTION OF THE DRAWINGS

FIG. 1 is a configuration diagram illustrating an apparatus for simultaneously measuring heterogeneous biosignals according to an exemplary embodiment of the present disclosure.

FIG. 2 is an explanation diagram illustrating a case in which a first sensing stage and a second sensing stage do not share electrodes.

FIG. 3 is an explanation diagram illustrating a case in which a first sensing stage and a second sensing stage share one electrode.

FIG. 4 is a configuration diagram illustrating a first sensing stage and a second sensing stage in the case of sharing one electrode.

FIG. 5 is an explanation diagram illustrating a case in which a second sensing stage measures a second biosignal by using a particular electrode substitute.

### DETAILED DESCRIPTION OF THE INVENTION

Hereinafter, an apparatus for simultaneously measuring heterogeneous biosignals and a measurement method thereof according to embodiments of the present disclosure will be described in detail with reference to the accompanying drawings.

It is noted that the embodiments of the present disclosure are illustrative of the present disclosure and do not limit the scope of the present disclosure. What can be easily inferred by those skilled in the art from the detailed description and embodiments of the present disclosure is interpreted as belonging to the scope of the present disclosure.

An apparatus 100 for simultaneously measuring heterogeneous biosignals according to the present disclosure is an apparatus for measuring two or more types of biosignals.

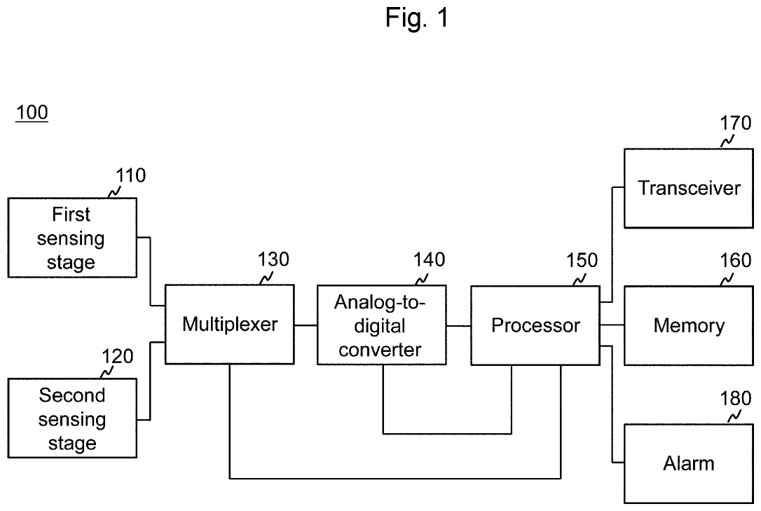

FIG. 1 is a configuration diagram illustrating an apparatus 100 for simultaneously measuring heterogeneous biosignals according to an exemplary embodiment of the present disclosure.

As shown in FIG. 1, the apparatus 100 for simultaneously measuring heterogeneous biosignals according to the exemplary embodiment of the present disclosure includes a first sensing stage 110, a second sensing stage 120, a multiplexer 130, an analog-to-digital converter 140, a processor 150, a memory 160, a transceiver 170, and an alarm 180.

The first sensing stage 110 may be realized as a circuit and measures a first biosignal. The first sensing stage 110 may be an electrocardiogram sensing stage measuring an electrocardiogram signal, for example.

Specifically, the first sensing stage 110 measures a first biosignal by using at least one electrode as a sensor. In the case in which the first sensing stage 110 is the electrocardiogram sensing stage, the voltage at a region where two electrodes are in contact with skin is measured using input signals from the two electrodes. That is, the electrocardiogram sensing stage measures the voltage stimulating heart muscles, in time series.

The second sensing stage 120 may be realized as a circuit and measures a second biosignal. The second sensing stage 120 may be a capacitive sensor measuring a respiratory signal, for example.

Specifically, the second sensing stage 120 measures a second biosignal by using at least one electrode or a particular electrode substitute as a sensor.

The multiplexer 130 receives the first biosignal from the first sensing stage 110 and the second biosignal form the second sensing stage 120, and outputs either the first biosignal or the second biosignal. That is, such two biosignals are integrated into one by the multiplexer 130 so that the analog-to-digital converter 140 coming after the multiplexer 130 is shared.

Specifically, the multiplexer 130 receives a selection signal from the processor 150, and selects and outputs either a first biosignal or a second biosignal. Herein, the multiplexer 130 can output the first biosignal and the second biosignal alternately in succession or non-alternatively based on sampling rate of the first biosignal and the second biosignal.

The analog-to-digital converter 140 converts an analog signal that is an output of the multiplexer 130 into a digital signal.

Generally, an electrocardiogram signal has a frequency of about 300 Hz, and a respiratory signal has a frequency of about 10 Hz. In the case of converting such two types of biosignals into digital signals by using one analog-to-digital converter 140, the analog-to-digital converter 140 needs to operate at the sampling rate that is two or more times higher than the frequency of an electrocardiogram signal, because electrocardiogram signal is higher than the respiratory signal. For this example, it is preferable to use the analog-to-digital converter 140 having the sampling rate of about 3 kHz.

Therefore, it is preferable that the multiplexer 130, which precedes the analog-to-digital converter 140, outputs the first biosignal and the second biosignal alternately, with the switching frequency the same as the sampling rate of the analog-to-digital converter 140.

That is, the multiplexer 130 outputs the first biosignal and the second biosignal alternately, and the analog-to-digital converter 140 outputs the first biosignal and the second biosignal, accordingly.

Herein, for convenience of description, although the multiplexer 130 and the analog-to-digital converter 140 have been described as separate constituents, an analog-to-digital converter including a multiplexer may be used as a substitute. That is, the multiplexer 130 and the analog-to-digital converter 140 may be provided as follows: the multiplexer 130 and the analog-to-digital converter 140 are separate constituents; or the multiplexer 130 and the analog-to-digital converter 140 are contained in one integrated circuit. For reference, the fact that the multiplexer 130 and the analog-to-digital converter 140 are provided as separate constituents means that the multiplexer 130 and the analog-to-digital converter 140 are a single element each or that the multiplexer 130 and the analog-to-digital converter 140 are separately provided in multiple circuit elements. In addition, the fact that the multiplexer 130 and the analog-to-digital converter 140 are contained in one integrated circuit means that the analog-to-digital converter which is a single element includes the multiplexer 130 or that one integrated circuit includes the multiplexer 130, the analog-to-digital converter 140, and other constituents, for example.

Alternatively, two analog-to-digital converters may be provided without a multiplexer.

The processor 150 performs signal processing on an output of the analog-to-digital converter 140, and performs separation of the first biosignal or the second biosignal for output. As the processor 150, a digital signal processor (DSP) may be used. The first biosignal and the second biosignal are alternately input to the processor 150. The processor 150 generates the selection signal for the multiplexer 130, so the processor 150 determines whether the signal input by the selection signal is the first biosignal or the second biosignal. That is, the processor 150 primarily determines whether a signal output from the analog-to-digital converter 140 is the first biosignal or the second biosignal. In addition, the processor 150 may control whether to make the analog-to-digital converter 140 perform signal conversion. That is, the processor 150 operates as a controller in terms of the multiplexer 130 and the analog-to-digital converter 140.

When the processor 150 determines that the signal output from the analog-to-digital converter 140 is the first biosignal, signal processing is performed using a first filter (not shown). Conversely, when the processor 150 determines that the signal output from the analog-to-digital converter 140 is the second biosignal, signal processing is performed using a second filter (not shown). Both the first filter and the second filter may be low-pass filters for removing noise, and may be realized by a digital filter in the form of a computer program by using the processor 150. In addition, since different biosignals are input to the first filter and the second filter, it is preferable that pass bands of the filters are also different.

In addition, it is preferable that the processor 150 adds, to the first biosignal or the second biosignal, time information on the time when the first or second biosignal is measured, and outputs the resulting biosignal. Specifically, the time information may be added by being received by the processor 150 from a real-time clock (RTC) (not shown). In addition, the measured time information may use the time when the biosignal is input to the processor 150 or the time when the biosignal is processed by the processor 150. This is because the time information measured at the first sensing stage 110 and the second sensing stage 120 is substantially equal to the time inputted to the processor 150 or to the time processed by the processor 150.

Herein, for convenience of description, the real-time clock (RTC) is described as being used, but time information may be used on the basis of the time when an external device receives the biosignal.

When it is determined that the first biosignal or the second biosignal is abnormal, it is preferable that the processor 150 adds an event mark of abnormality to an abnormal part of the first biosignal or the second biosignal and outputs the resulting biosignal. Preferably, the event mark of abnormality added to the abnormal part of the first biosignal or the second biosignal includes event information so that a type of abnormality is recognized.

In the case in which the first biosignal is an electrocardiogram signal, the processor 150 may determine whether the electrocardiogram signal is normal, by using whether all the P, Q, R, S, and T waves are present, whether the P, Q, R, S, and T waves are correct in order, whether the P, Q, R, S, and T waves are correct (or regular) in shape, whether intervals of the Q, R, and S waves are regular, or whether the period of the electrocardiogram signal is within a specified range. In addition, in the case in which the second biosignal is a respiratory signal, the processor 150 determines whether the respiratory signal is normal, by using the respiratory rate, etc.

In addition, when it is determined that both the first biosignal and the second biosignal are abnormal simultaneously or within a predetermined period of time, the processor 150 adds respective event marks of complex abnormality both to an abnormal part of the first biosignal and to an abnormal part of the second biosignal and outputs the resulting biosignals. Whether both the first biosignal and the second biosignal are normal or abnormal simultaneously or within a predetermined period of time can be determined by using the heart rate and the respiratory rate. For example, when both the respiratory rate and the heart rate are fast, it is determined that the first biosignal and the second biosignal are normal. When the respiratory rate is slow but the heart rate is fast, it is determined that the first biosignal and the second biosignal are abnormal. In addition thereto, there are several situations in which it is determined that the first biosignal and the second biosignal are abnormal. However, even when it is determined that the first biosignal and the second biosignal are abnormal, the categories, such as abnormality, or demand for continuous monitoring, etc., are variously expressed with different types of event marks. That is, it is preferable that the respective event marks of complex abnormality in the first biosignal and the second biosignal include event information so that types of the abnormality are recognized.

Preferably, signal processing by the processor 150 is performed in this order: processing the first biosignal and second biosignal by using the first filter and the second filter, respectively; and next, adding the time information and various types of event marks to the first biosignal and the second biosignal that are separated.

The memory 160 stores therein the first biosignal or the second biosignal processed by the processor 150. In addition, the transceiver 170 transmits the first biosignal or the second biosignal processed by the processor 150 to an external device, and receives a signal from the external device. The transceiver 170 can be various types of wired or wireless communications.

Specifically, when a communication state using the transceiver 170 is stable, the first biosignal or the second biosignal is transmitted to an external device through the transceiver 170. Conversely, when the communication state using the transceiver 170 is unstable, the first biosignal or the second biosignal is stored in the memory 160. In addition, when the communication state using the transceiver 170 is changed from an unstable state to a stable state, it is preferable that the data stored in the memory 160 is transmitted to an external device through the transceiver 170. The processor 150 may determine whether the communication state using the transceiver 170 is stable. Because storing in the memory 160 only is made when the communication state using the transceiver 170 is unstable, the memory 160 having a small capacity may be used.

In addition, the characteristics of the first sensing stage 110, the second sensing stage 120, the multiplexer 130, the analog-to-digital converter 140, the processor 150, the memory 160, and the alarm 180 may be changed through the transceiver 170. As a simple example, the first sensing stage 110 is activated and the second sensing stage 120 is deactivated.

The alarm 180 outputs a visual or auditory warning signal. The alarm 180 may be realized using various devices, such as a buzzer, or a light-emitting element. In addition, preferably, the alarm 180 outputs a warning signal in a preset manner according to the event mark added to the first biosignal or the second biosignal. That is, preferably, depending on the event mark added to the first biosignal or the second biosignal, different warning signals are output. Furthermore, the processor 150 performs control such that the alarm 180 outputs a warning signal.

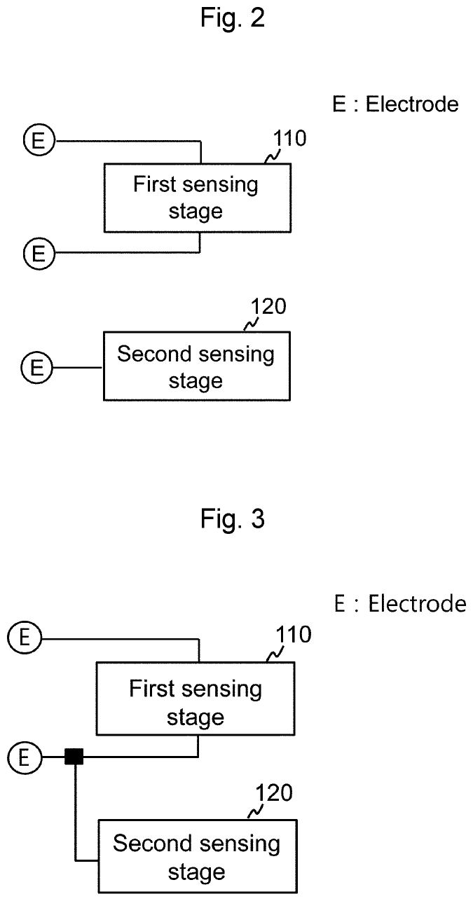

FIG. 2 is an explanation diagram illustrating a case in which a first sensing stage 110 and a second sensing stage 120 do not share electrodes.

That is, as shown in FIG. 2, in the present disclosure, the first sensing stage 110 and the second sensing stage 120 may use different electrodes, not sharing electrodes.

In the case in which an apparatus 100 for simultaneously measuring heterogeneous biosignals according to an exemplary embodiment of the present disclosure is realized in the form of a patch, it is expected to be difficult to reduce the patch in size when all multiple electrodes are used. Therefore, it is preferable that the first sensing stage 110 and the second sensing stage 120 share at least one electrode. Alternatively, the second sensing stage 120 may measure the second biosignal by using an electrode substitute rather than an electrode, thereby reducing the patch in size.

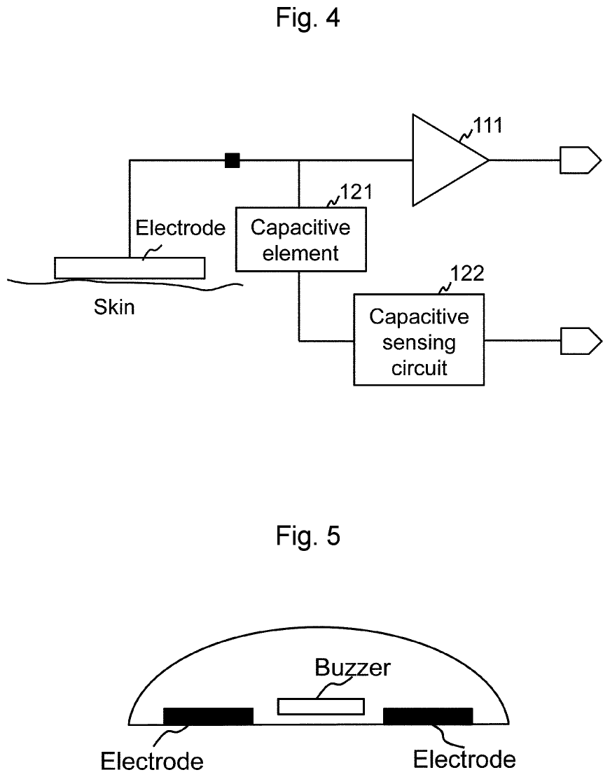

FIG. 3 is an explanation diagram illustrating a case in which a first sensing stage 110 and a second sensing stage 120 share one electrode. FIG. 4 is a configuration diagram illustrating the first sensing stage 110 and the second sensing stage 120 in the case of sharing one electrode.

Specifically, considering that the first biosignal is an electrocardiogram signal and the second biosignal is a respiratory signal, one electrode may be shared. This case may be applied to operating the first sensing stage 110 and the second sensing stage 120 in a time division manner.

The first sensing stage 110 includes an amplifier 111 that receives and amplifies a signal of at least one electrode. Specifically, as the amplifier 111, it is preferable to use a differential signal amplifier that receives and amplifies a signal of a non-shared electrode as well as the shared electrode. In addition, the amplifier 111 may use a voltage amplifier having a high input impedance.

It is preferable that the second sensing stage 120 includes: a capacitive element 121 connected to the electrode that is shared with the first sensing stage 110; and a capacitive sensing circuit 122 connected to the capacitive element 121 and sensing a capacitance value. Herein, the capacitive element 121 refers to a simple capacitor element as well as an electronic element capable of representing a capacitance value. In addition, the capacitive element 121 may use either a capacitor or a capacitor equivalent device such as an electro-static discharge (ESD) protection device and buzzer. For reference, the capacitive sensing circuit 122 may be realized by various methods, as can be found through the Internet or search for patents.

In general, an ESD protection device enters a turn-on state when a high voltage is applied, but is not turned on when a low voltage is applied and operates as a capacitor.

For reference, the capacitance caused by the shared electrode and the capacitive element are connected in series in an equivalent circuit, and a series capacitance value is measured by the capacitive sensing circuit 122 connected to the capacitive element 121.

FIG. 5 is an explanation diagram illustrating a case in which the second sensing stage 120 measures a second biosignal by using a particular electrode substitute.

Specifically, FIG. 5 is a cross-sectional view of an apparatus for simultaneously measuring heterogeneous biosignals, the apparatus being realized as a patch.

Considering that the first biosignal is an electrocardiogram signal and the second biosignal is a respiratory signal, the second sensing stage 120 may be connected to a buzzer for outputting a warning signal. In the case of the buzzer, the buzzer is not in direct contact with the skin of a subject, but buzzer size is large enough. Thus, a capacitance value between the buzzer and the skin changes depending on respiration. Accordingly, the second sensing stage 120 may measure the second biosignal using the change in the capacitance value between the buzzer and the skin of the subject.

A method of simultaneously measuring heterogeneous biosignals according to an exemplary embodiment of the present disclosure uses the apparatus 100 for simultaneously measuring heterogeneous biosignals of the present disclosure described above. Therefore, it is noted that the method includes all features of the apparatus 100 for simultaneously measuring heterogeneous biosignals of the present disclosure, even if there is no particular description.

According to an exemplary embodiment of the present disclosure, there is provided a method of simultaneously measuring heterogeneous biosignals, the method including: receiving a first biosignal and a second biosignal, and outputting either the first biosignal or the second biosignal at step S10; converting an analog signal of step S10 into a digital signal at step S20; receiving the digital signal of step S20 and performing signal processing thereon at step S30; transmitting a signal output at step S30 to an external device at step S40; and storing the signal output at S30 in the memory 160 at step S50 when transmission of the signal at step S40 is difficult.

Specifically, at step S10, either the first biosignal or the second biosignal is selected using a selection signal and is output, and the first biosignal and the second biosignal are output alternately in succession or non-alternatively based on sampling rate of the first biosignal and the second biosignal.

Furthermore, step S30 includes: performing, when the first biosignal is input, filtering on the first biosignal by using a first filter at step S31; performing, when the second biosignal is input, filtering on the second biosignal by using a second filter at step S32; adding measured time information to the filtered first biosignal of step S31 at step S33; adding measured time information to the filtered second biosignal of step S32 at step S34; adding, when it is determined that the filtered first biosignal of step S31 is abnormal, an event mark of abnormality to an abnormal part of the first biosignal at step S35; adding, when it is determined that the filtered second biosignal of step S32 is abnormal, an event mark of abnormality to an abnormal part of the second biosignal at step S36; and adding, when it is determined that both the filtered first biosignal of step S31 and the filtered second biosignal of step S32 are abnormal simultaneously or within a predetermined period of time, respective event marks of complex abnormality to an abnormal part of the first biosignal and to an abnormal part of the second biosignal at step S37.

However, step S35 and step S33 may be performed simultaneously or in parallel, or step S35 may be performed after step S33. Similarly, step S36 and step S34 may be performed simultaneously or in parallel, or step S36 may be performed after step S34. In addition, step S37, step S35, and step S36 may be performed simultaneously or in parallel, or step S37 may be performed after step S35 and step S36.

In addition, the method of simultaneously measuring heterogeneous biosignals according to an exemplary embodiment of the present disclosure further includes measuring the first biosignal and the second biosignal at step S05 before step S10. The first biosignal and the second biosignal measured at step S05 are input to step S10. The first biosignal and the second biosignal may be measured by sharing at least one electrode.

Specifically, the second biosignal is measured by sensing a capacitance value using a node connected to the capacitive element 121. In addition, preferably, the capacitive element 121 is connected to the electrode shared to measure the first biosignal and the second biosignal.

That is, the capacitance caused by the shared electrode and the capacitive element are connected in series in an equivalent circuit, and a series capacitance value is measured by using the node connected to the capacitive element 121.

In addition, according to another embodiment, the second biosignal may be measured using a change in the capacitance value between a buzzer for outputting a warning signal and the skin of a subject.

As described above, according to the apparatus 100 for simultaneously measuring heterogeneous biosignals and the measurement method thereof of the present disclosure, the apparatus can be realized in the form of a patch reduced in size by sharing some of constituents when heterogeneous biosignals are simultaneously measured.

What is claimed is:

1. An apparatus for measuring biosignals, the apparatus comprising:
a multiplexer configured to receive a first biosignal and a second biosignal, and to output either the first biosignal or the second biosignal;
an analog-to-digital converter configured to convert an analog signal that is an output of the multiplexer into a digital signal; and
a processor configured to perform signal processing on an output of the analog-to-digital converter.

2. The apparatus of claim 1, wherein the multiplexer selects and outputs either the first biosignal or the second biosignal, and outputs the first biosignal and the second biosignal alternately in succession.

3. The apparatus of claim 1, wherein the processor performs signal processing by using a first filter when the first biosignal is input, or performs signal processing by using a second filter when the second biosignal is input.

4. The apparatus of claim 1, further comprising:
a memory configured to store therein the first biosignal or the second biosignal processed by the processor; and
a transceiver configured to transmit the first biosignal or the second biosignal processed by the processor to an external device.

5. The apparatus of claim 4, wherein the apparatus transmits the first biosignal or the second biosignal to the external device through the transceiver when a communication state using the transceiver is stable, or stores the first biosignal or the second biosignal in the memory when the communication state using the transceiver is unstable.

6. The apparatus of claim 4, wherein when it is determined that the first biosignal or the second biosignal is abnormal, the processor adds an event mark of abnormality to an abnormal part of the first biosignal or the second biosignal.

7. The apparatus of claim 6, wherein when it is determined that both the first biosignal and the second biosignal are abnormal simultaneously or within a predetermined period of time, the processor adds respective event marks of complex abnormality to an abnormal part of the first biosignal and to an abnormal part of the second biosignal.

8. The apparatus of claim 1, further comprising:
a first sensing stage configured to measure the first biosignal by using at least one electrode; and
a second sensing stage configured to measure the second biosignal by using at least one electrode,
wherein the first sensing stage and the second sensing stage share at least one electrode, and
wherein the multiplexer configured to receive the first biosignal from the first sensing stage and the second biosignal from the second sensing stage.

9. The apparatus of claim 8, wherein the first biosignal is an electrocardiogram signal,
the second biosignal is a respiratory signal, and
the first sensing stage and the second sensing stage share the one electrode.

10. The apparatus of claim 9, wherein the second sensing stage comprises:
a capacitive element connected to the electrode shared with the first sensing stage; and
a capacitive sensing circuit connected to the capacitive element and configured to sense a capacitance value.

11. The apparatus of claim 10, wherein the capacitive element uses either a capacitor or an ESD protection device.

12. The apparatus of claim 1, further comprising:
a first sensing stage configured to measure the first biosignal by using at least one electrode; and
a second sensing stage configured to measure the second biosignal,
wherein:
the first biosignal is an electrocardiogram signal;
the second biosignal is a respiratory signal;
the second sensing stage is connected to a buzzer, and measures the second biosignal using a change in a capacitance value between the buzzer and a skin of a subject; and
the multiplexer configured to receive the first biosignal from the first sensing stage and the second biosignal from the second sensing stage.

13. A method of measuring biosignals, the method comprising:
(b) receiving a first biosignal and a second biosignal, and outputting either the first biosignal or the second biosignal;
(c) converting an analog signal of the step (b) into a digital signal; and
(d) receiving the digital signal output at the step (c) and performing signal processing thereon.

14. The method of claim 13, wherein at the step (b), either the first biosignal or the second biosignal is selected and output, and the first biosignal and the second biosignal are output alternately in succession.

15. The method of claim 13, wherein the step (d) comprises:
(d-1) performing, when the first biosignal is input, filtering on the first biosignal by using a first filter;
(d-2) performing, when the second biosignal is input, filtering on the second biosignal by using a second filter;
(d-3) adding measured time information to the filtered first biosignal of the step (d-1); and
(d-4) adding measured time information to the filtered second biosignal of the step (d-2).

16. The method of claim 15, wherein the step (d) further comprises:
(d-5) adding, when it is determined that the filtered first biosignal of the step (d-1) is abnormal, an event mark of abnormality to an abnormal part of the first biosignal;
(d-6) adding, when it is determined that the filtered second biosignal of the step (d-2) is abnormal, an event mark of abnormality to an abnormal part of the second biosignal; and
(d-7) adding, when it is determined that both the filtered first biosignal of the step (d-1) and the filtered second biosignal of the step (d-2) are abnormal simultaneously or within a predetermined period of time, respective event marks of complex abnormality to an abnormal part of the first biosignal and to an abnormal part of the second biosignal.

17. The method of claim 13, further comprising:
(e) transmitting a signal output at the step (d) to an external device; and
(f) storing the signal output at the step (d) in a memory when transmission of the signal at the step (e) is difficult.

18. The method of claim 13, further comprising:
(a) measuring the first biosignal and the second biosignal,
wherein the first biosignal and the second biosignal are measured by sharing at least one electrode at the step (a), and
wherein the first biosignal and the second biosignal measured at the step (a) are input to the step (b).

19. The method of claim 18, wherein the second biosignal is measured by sensing a capacitance value using a node connected to a capacitive element,
wherein the capacitive element is connected to the electrode shared to measure the first biosignal and the second biosignal.

20. The method of claim 13, further comprising:
(a) measuring the first biosignal and the second biosignal,
wherein the second biosignal is measured using a change in a capacitance value between a buzzer and a skin of a subject at the step (a), and
wherein the first biosignal and the second biosignal measured at the step (a) are input to the step (b).
